(19) United States
(12) Patent Application Publication
(10) Pub. No.: US 2004/0249427 A1
(43) Pub. Date: Dec. 9, 2004

(54) Medical cooler device

(76) Inventor: Yunes Nabilsi, Artesia, CA (US)

Correspondence Address: LAW OFFICES OF CLEMENT CHENG, 17220 NEWHOPE STREET #127, FOUNTAIN VALLEY, CA, 92708, US

(21) Appl. No.: 10/456,059

(22) Filed: Jun. 6, 2003

(51) Int. Cl. A61F 7/00

(52) U.S. Cl. 607/104; 607/108; 607/109; 607/112

(57) ABSTRACT

A portable medical cooling device has two circuits of heat transfer liquid, a hot circuit and a cold circuit. The cold circuit begins at a patient's body conducting heat to a cooling pad cool heat exchanger. The cooling pad conforms to a specific part of a patient's body. The airflow leaves the housing via an exhaust port formed on the housing allowing exhausting of hot air into an ambient environment. The cold circuit completed by return of heat transfer liquid by cool tubing to the cooling pad via pressure created by a cold circuit pump. The hot circuit is completed by return of heat transfer liquid by cool tubing to the hot face via pressure created by a hot circuit pump and a thermostat controls voltage of the thermoelectric element to achieve and maintain a user defined temperature in the cold circuit heat transfer liquid.

### BACKGROUND OF THE INVENTION

Patients often need postoperative care especially after cosmetic surgery. To have a good result patients require postoperative cooling therapy to ease inflammation and swelling. After surgery, some patients use compression stockings on the legs, but this is often unavailable for areas such as eyes.

Traditionally, hospitals apply an ice bag or cold gel pack to the area suffering from swelling. In any emergency, a nurse can get a rubber surgical glove filled with crushed ice, and place them on the treated area. However, the shape of a glove stuffed with crushed ice is not flexible enough to conform the shape of the treated area, and the ice in the glove would not last through the night.

Patients must wake from bed at least once during midnight apply a new ice bag or cold gel pack. If the treated areas were in delicate parts such as the eyes, waking from bed would be torment. The shape and weight of an ice bag would not be flexible enough to conform to the treated area. The weight of the ice bag is often too heavy and presses the wound to make more swelling, pain and suffering.

Various devices have been used in medicine to cool patients. U.S. Pat. No. 5,894,615 to Alexander shows a temperature selectively controllable supporting pad for a patient to lay upon.

### BRIEF DESCRIPTION OF DRAWINGS

FIG. 1(a) is a front elevational view of the cooling unit of the present invention

FIG. 1(b) is a top elevational view of the cooling unit of the present invention

FIG. 2 is a tubing chart demonstrating the tubing connections from the cooling unit to the cooling attachment of the present invention

FIG. 3(a) is a front elevational view of the first implement of the cooling attachment as an eye pad.

FIG. 3(b) is a perspective front view of the same eye pad shown on FIG. 3, being disposed over the eyes and nose of the wearer.

FIG. 4 is a front elevational view of the second implement of the cooling attachment as a mask pad.

FIG. 5 is a front elevational view of the third implement of the cooling attachment as a chin pad of the present invention.

FIG. 6 is a front elevational view of the forth implement of the cooling attachment as a chest pad.

FIG. 7 is a perspective side view of the fifth implement of the cooling attachment as an elbow pad, being disposed on the elbow of the wearer.

FIG. 8(a) is a front elevational view of the sixth implement of the cooling attachment as a knee pad.

FIG. 8(b) is a perspective front view of the same knee pad shown on FIG. 8(a), being disposed over the knee of the wearer.

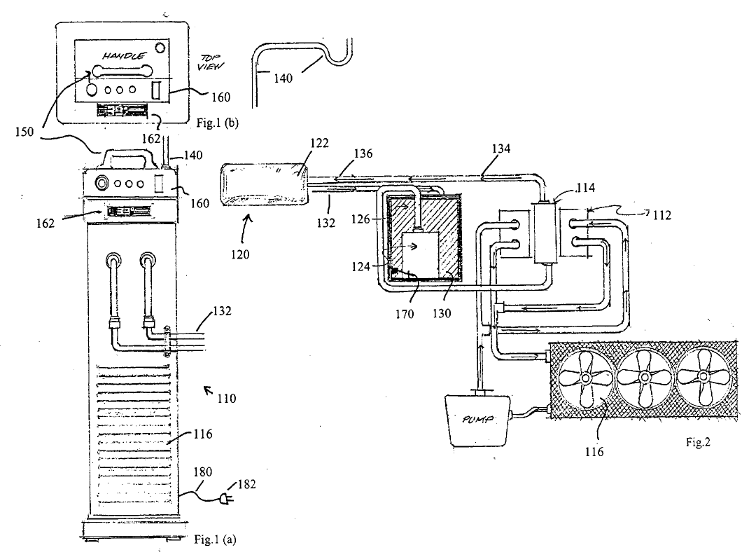

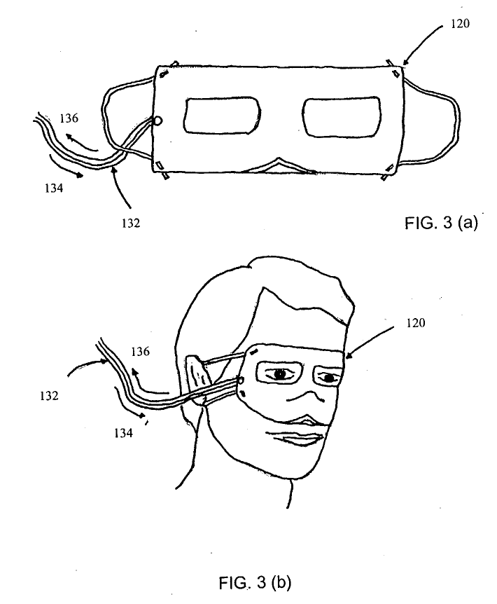

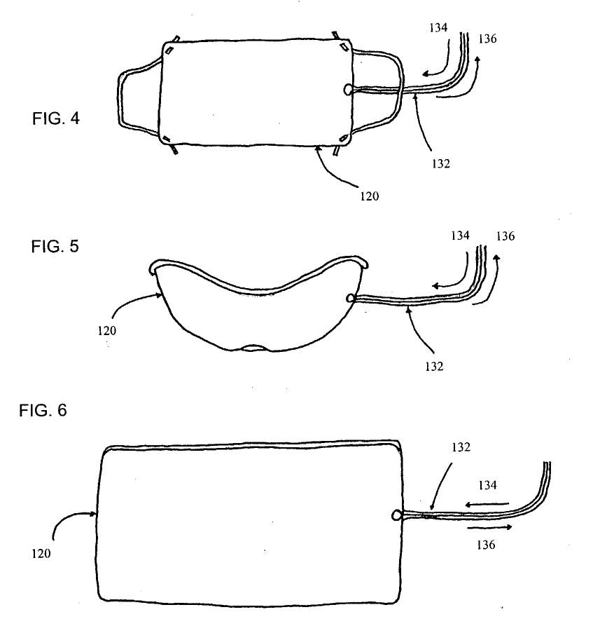

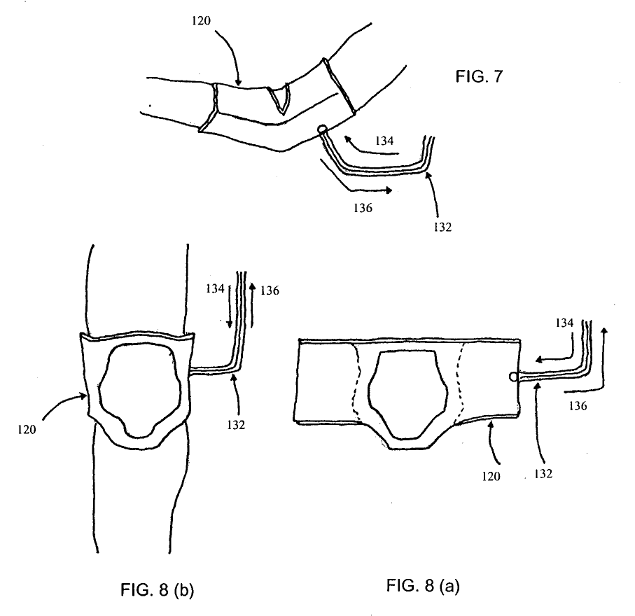

### OBJECT OF THE INVENTION

The present invention attempts to ease the suffering and discomfort caused by a post surgery patient's inflammation and swelling by providing a constant temperature cooling pad adaptable to specific body parts. The present invention keeps a patient from taking midnight trips to get new cool pads.

### DETAILED DESCRIPTION OF THE PREFERRED EMBODIMENT

### Summary of Invention

The present invention is designed to provide surgical patients a comforting post-surgery cool therapy treatment. The present invention can keep a cooling pad (120) at approximately 37 degrees Fahrenheit constantly, by using a pump and a tube pair connected to a cold fluid supplied to transmit and exchange temperature in the cooling pad (120). The patient can use a bedside keypad to control his or her desired temperature. Furthermore, the cooling pad (120) is lightweight and soft, conforming to particular areas to keep the operated area cool and reduce swelling without frost bite risk.

The present invention can be used at home, in clinics and hospitals wherever cool therapy is needed. The device comprises a cooling unit (110), a pair of tubes, and a cooling pad (120). The cooling pad (120) is a cool heat exchanger thermally coupled to a person's skin. The cooling unit (110) has both a cool heat exchanger (114) and a warm heat exchanger (112).

### Cooling Pad

Chilled liquid has a closed loop system such that it recycles within the device between the cooling pad (120) and the cooling unit cool heat exchanger (114). A patient does not need to replace the pad on the patient's body. When a patient is fully recuperated, the device is removed and may be moved to help the recuperation of a different patient. A different patient may have a different body part requiring cooling and may have a different cooling requirement. The cooling pad unit is modular and interchangeable via the quick connection means in the tubing pair (132) leading from the cooling pad (120).

The cooling pad (120) has a soft surface and is shaped to conform to a person's body. The pad is hollow and holds a flow of cool liquid such as water. Upon being placed on a patient, the cooling pad (120) transfers heat away from a person's body. The cooling pad (120) can be formed of plastic in several configurations. It can be shaped in the form of a mask for cooling a person's eyes, shaped in the form of a sleeve for the cooling of a patient's limbs, or shaped in the form of a collar for attachment to a patient's neck and chin area. A number of different types of cool heat exchangers can be used in conjunction with one single cooling unit (110). The cooling pads may be interchanged by a quick connect feature built into a tubing pair (132).

Figure three (a) shows an eye mask having a cool tubing (134) to cooling pad (120) and a warm tubing (136) to cooling unit. The mask has eye holes and each of these can fit over a person's face as shown in figure three (b). Made of soft fabric, the mask contains a flow of water or other heat transfer liquid (130).

Figure four shows a cooling pad (120) having a pair of straps allowing attachment to a patient. A patient may use the cooling pad (120) on a body part such as a forearm or on the abdomen. Figure five shows a cooling pad shaped to fit a person's chin. Figure six shows a larger pad (120) that can be draped over a person's abdomen or chest area. Figure seven shows a person wearing the cooling pad (120) around an elbow. Figure eight (a) shows an adapted to fit around a person's knee. Figure eight (b) shows a person wearing a knee cooling pad (120). The knee cooling pad (120) optionally has an insulation pad portion on the kneecap to prevent heat gain on the kneecap area.

Cooling pads additionally include an inlet and an outlet for routing the cool liquid. The cool liquid can be either water or a mixture of both water and alcohol. Cooling pads are nonstick, soft and drape over a person's body. A retaining arm stand similar to an IV bag stand holds the hot and cold line tubing pair (132) traveling to cooling pad (120). Additionally, the cooling pad (120) may include a thermal reservoir in the form of a gel pack that is formed around a portion of the cooling pad (120). The cooling pad (120) may consist of a laminated plastic bag formed from layers of plastic so that a thermal reservoir may be formed within one of the laminated regions. Various laminated regions may include a thermal gel material capable of retaining greater heat than the circulating heat transfer liquid (130). It is preferable to enclose the cooling pad (120) in a cloth sleeve so that skin does not directly contact the plastic material. The cooling pad cool heat exchanger (122) includes an inlet and an outlet to the tubing pair (132).

### Cooling Unit

The cooling pad (120) can be attached to a cooling unit (110). The cooling unit (110) contains a thermoelectric Peltier element having a cold face and a hot face. The hot face interfaces with the hot cycle and the cold face interfaces with the cold cycle. The cooling unit (110) has a warm thermo-electrically driven internal heat exchanger and a cool internal heat exchanger firmly coupled to the hot and cold face of the thermoelectric element. A cold cycle and a hot cycle are contained within the cooling unit (110). The cooling pad (120) is interchangeable so that a variety of specialized shape cooling pad (120) modules can interchange by quick connect couplings depending upon the part of the body to be cooled. The cooling unit (110) uses an ordinary electrical current operating at 110 volts. The cooling unit (110) additionally includes an exhaust allowing exhaust of warm air from the cooling unit (110).

The inlet is attached to an inlet tubing connector. The outlet is attached to an outlet tubing connector. A patient lying on a bed can use the cool pad without changing the cool pad. The flow of heat transfer liquid (130) continually draws heat from the cooling pad (120) and patient.

The cycle of cool water or liquid is driven by a pump. A cool water reservoir (126) is located within the cooling unit (110) and adds thermal capacitance to the cool water cycle. The cool cycle pump (124) is preferably located within the cool water reservoir (126) allowing cooling of the pump by liquid returning from the patient's cooling pad (120). The cool cycle pump (124) circulates water to the thermoelectric cooler's cool heat exchangers for cooling.

The hot cycle pump circulates the heated liquid in the separate cycle of hot heat transfer liquid (130). The hot cycle heat transfer liquid travels through the hot heat exchangers of the thermoelectric cooler and receives heat from the hot heat exchangers. The hot face of the thermoelectric device heats the heat transfer liquid (130). The hot heat transfer liquid cycles to a radiator having fans to air cool the radiator. After the radiator, the hot cycle pump circulates the heat transfer liquid (130) back to the hot heat exchangers of the thermoelectric cooler.

### Controls

The temperature of the cooling pad (120) may be maintained at a constant temperature by adjusting a thermostat (160). The thermostat (160) regulates the temperature of cool liquid flowing from the cooling unit (110) to the cooling pad (120). The cooling pad (120) may contain a thermometer (170), or the thermometer may be placed within the cool cycle of the cooling unit (110). The cooling unit thermometer (170) may also be used in conjunction with a cooling pad thermometer. The best method is to have a cooling unit thermometer measuring temperature of the chilled liquid leaving the cooling unit (110). The thermostat temperature and the temperature of the chilled liquid leaving the cooling unit cool heat exchanger (114) are both displayed in the display on the cooling unit (110). The display is preferably a large LCD display (162). The controls comprise a plurality of buttons and knobs near the upper portion of the cooling unit (110) by the large LCD display (162).

A computer may control the thermostat (160) electronically so that the temperature of the cooling pad (120) changes according to a pre-programmed schedule. The schedule may depend upon the type of patient being treated. Some patients may require a short period of cooling and some patients may require a longer period of cooling.

A control system increases the thermoelectric cooling power in proportion to the difference of temperature between the thermostat (160) and thermometer (170). The cooling unit (110) may have an array of thermal electric coolers in series. The computer may also vary the voltage of the fans that cool the hot heat exchanger and the power of the hot and cold cycle pump circulating the hot and cold cycle.

Alternatively, the thermoelectric element (TEC) may be implemented in a series of multiple TEC elements. The thermostat and temperature controller means may activate or deactivate a number of elements to maintain a certain preset temperature. Discrete activating and deactivating a number of elements simplifies the temperature controller means, also called a thermostat and does not require variance of voltage in any of the components such as pumping mechanism, fans, or thermoelectric element. For example, when the temperature difference between the thermometer or thermocouple and the desired temperature is great, all of the TEC elements may be activated. When the desired temperature difference is smaller, some of the TEC elements may be deactivated.

### Housing

A handle (150) formed at the top of the cool unit allows a user to carry the cooling unit (110). The cooling unit (110) is housed within small rectangular box. The cooling unit (110) has a chilled liquid tubing leaving the cool heat exchangers in the cooling unit (110) traveling to the cool heat exchangers in the pad unit. The warm return tubing is also flexible and is slightly warmer, but can be routed alongside the chilled liquid tubing.

The pair of tubes are maintained in position above a patient by a stand holding the tubes. The only part of the device necessarily touching the user is the cooling pad (120) unit. The stand is attached to the cooling unit (110) for stability and telescopes from a contracted position to an expanded position. The telescoping stand (140) maintains a constant height adjustable by a user. Because of the cooling unit (110)'s weight, the telescoping stand (140) is maintained in a stable position.

### Call Out List of Elements

110 Cooling Unit

112 Warm Heat Exchanger Of Cooling Unit

114 Cool Heat Exchanger Of Cooling Unit

116 Exhaust Port Of Cooling Unit

120 Cooling Pad

122 Cool Heat Exchanger Of Cooling Pad

124 Cool Pump

126 Reservoir

130 Heat Transfer Liquid

132 Tubing Pair For Liquid

134 Cool Tubing To Cooling Pad

136 Warm Tubing To Cooling Unit

140 Telescoping Stand For Holding Tubing Pair

150 Handle For Cooling Unit

160 Thermostat

162 LCD Temperature Readout

170 Thermometer

180 Electrical Power Cord

182 Electrical Power Plug

1. A portable medical cooling device comprising: two circuits of heat transfer liquid, a hot circuit and a cold circuit; wherein the cold circuit begins at a patient's body conducting heat to a cooling pad cool heat exchanger, the cooling pad holding a constant stream of heat transfer liquid, the cooling pad shaped to conform to a specific part of a patient's body, the heat from the patient's body traveling by a constant stream of heat transfer liquid from the cooling pad to a warm tubing to a cooling unit, the cold circuit heat transfer liquid entering a housing holding a cooling unit comprising a thermoelectric element having a cold face receiving heat by convection from the cold circuit, and a hot face transferring heat by convection to the hot circuit then to a warm heat exchanger on the hot circuit which receives airflow generated by a fan, the airflow leaves the housing via an exhaust port formed on the housing allowing exhausting of hot air into an ambient environment, the cold circuit completed by return of heat transfer liquid by cool tubing to the cooling pad via pressure created by a cold circuit pump, the hot circuit completed by return of heat transfer liquid by cool tubing to the hot face via pressure created by a hot circuit pump, wherein a thermostat controls voltage of the thermoelectric element to achieve and maintain a user defined temperature in the cold circuit heat transfer liquid, wherein an LCD temperature readout display shows the desired temperature setting and the temperature of a thermometer reading.

2. The portable medical cooling device of claim 1 further comprising a reservoir in the cooling unit holding heat transfer liquid returning from the cooling pad through the warm tubing.

3. The portable medical cooling device of claim 1 wherein warm tubing and cool tubing is consolidated into a single conduit to form a tubing pair for liquid.

4. The portable medical cooling device of claim 2 further comprising a telescoping stand for holding the tubing pair so that it does not touch the patient.

5. The portable medical cooling device of claim 1 further comprising a cooling unit handle.

6. The portable medical cooling device of claim 1 wherein the heat transfer liquid is water.

7. The portable medical cooling device of claim 1 wherein the cooling pad is shaped in the form of a mask for cooling a person's eyes, wherein eye holes fit over a person's eyes.

8. The portable medical cooling device of claim 1 wherein the cooling pad is shaped in the form of a sleeve for the cooling of a patient's limbs.

9. The portable medical cooling device of claim 1 wherein the cooling pad is shaped in the form of a collar for attachment to a patient's chin area.

10. The portable medical cooling device of claim 1 wherein the cooling pad is shaped in the form of a sleeve for attachment to a patient's torso area.

11. The portable medical cooling device of claim 1 wherein the cooling pad is shaped in the form of a jointed sleeve for attachment to a patient's elbow area, wherein a person may wear the cooling pad around an elbow.

12. The portable medical cooling device of claim 1 wherein the cooling pad is shaped in the form of a jointed sleeve for attachment to a patient's knee area, wherein a person may wear the cooling pad around a knee.

13. The portable medical cooling device of claim 1 wherein the cooling pad is shaped in the form of a jointed sleeve for attachment to a patient's knee area, wherein a person may wear the cooling pad around a knee, wherein the knee cooling pad has an insulation pad portion on the kneecap to prevent heat gain on the kneecap area.

14. A portable medical cooling device comprising: a cooling unit cooling a flow of heat transfer liquid, a hollow cooling pad holding a flow of heat transfer liquid, wherein the heat from the patient's body travels by a, constant stream of heat transfer liquid from the cooling pad to a warm tubing to the cooling unit, wherein the cooling pad is a shaped to conform to a specific part of a patient's body.

15. The portable medical cooling device of claim 14 wherein the cooling pad is shaped in the form of a mask for cooling a person's eyes, wherein eye holes fit over a person's eyes.

16. The portable medical cooling device of claim 14 wherein the cooling pad is shaped in the form of a sleeve for the cooling of a patient's limbs.

17. The portable medical cooling device of claim 14 wherein the cooling pad is shaped in the form of a collar for attachment to a patient's chin area.

18. The portable medical cooling device of claim 14 wherein the cooling pad is shaped in the form of a sleeve for attachment to a patient's torso area.

19. The portable medical cooling device of claim 14 wherein the cooling pad is shaped in the form of a jointed sleeve for attachment to a patient's elbow area, wherein a person may wear the cooling pad around an elbow.

20. The portable medical cooling device of claim 14 wherein the cooling pad is shaped in the form of a jointed sleeve for attachment to a patient's knee area, wherein a person may wear the cooling pad around a knee.

21. The portable medical cooling device of claim 14 wherein the cooling pad is shaped in the form of a jointed sleeve for attachment to a patient's knee area, wherein a person may wear the cooling pad around a knee, wherein the knee cooling pad has an insulation pad portion on the kneecap to prevent heat gain on the kneecap area.

22. A portable medical cooling device comprising: a thermoelectric Peltier cooling unit cooling a constant isothermal flow of heat transfer liquid determined by a thermostat, a hollow cooling pad holding a flow of heat transfer liquid, wherein the heat from the patient's body travels by a constant isothermal stream of heat transfer liquid from the cooling pad through a tubing pair to the cooling unit, wherein the cooling pad is shaped to conform to a specific part of a patient's body, wherein the cooling pad fits inside a fabric sleeve shaped to conform to a specific part of a patient's body and having straps that allow attachment to a specific part of a patient's body.

23. The portable medical cooling device of claim 22 wherein the cooling pad is shaped in the form of a mask for cooling a person's eyes, wherein eye holes fit over a person's eyes.

24. The portable medical cooling device of claim 22 wherein the cooling pad is a shaped in the form of a jointed sleeve for attachment to a patient's elbow area, wherein a person may wear the cooling pad around an elbow.

25. The portable medical cooling device of claim 22 wherein the cooling pad is shaped in the form of a jointed sleeve for attachment to a patient's knee area, wherein a person may wear the cooling pad around a knee.

26. The portable medical cooling device of claim 22 wherein the cooling pad is shaped in the form of a sleeve for the cooling of a patient's limbs.

27. The portable medical cooling device of claim 22 wherein the cooling pad is shaped in the form of a collar for attachment to a patient's chin area.

28. The portable medical cooling device of claim 22 wherein the cooling pad is shaped in the form of a sleeve for attachment to a patient's torso area.
